(19) United States
(12) Patent Application Publication
(10) Pub. No.: US 2009/0226916 A1
(43) Pub. Date: Sep. 10, 2009

(54) Automated Analysis of DNA Samples

(75) Inventors: Bruce E. DeSimas, Danville, CA (US); Ravi Gupta, Foster City, CA (US); Lisa M. Calandro, San Ramon, CA (US)

Correspondence Address: LIFE TECHNOLOGIES CORPORATION;C/O INTELLEVATE, P.O. BOX 52050, MINNEAPOLIS, MN, 55402, US

(73) Assignee: Life Technologies Corporation, Carlsbad, CA (US)

(21) Appl. No.: 12/364,447

(22) Filed: Feb. 2, 2009

Related U.S. Application Data
(60) Provisional application No. 61/063,173, filed on Feb. 1, 2008.
(60) Provisional application No. 61/038,975, filed on Mar. 24, 2008.

(51) Int. Cl. C12Q 1/68; C12M 1/34

(52) U.S. Cl. 435/6; 435/287.2

(57) ABSTRACT

The present invention provides a system and methods for deconvoluting mixed DNA samples. Applications developed according to the invention may be used for resolving two or more person mixtures into easy to interpret contributor profiles and to perform automated statistical calculations. An automated analysis approach for mixed samples integrating hardware and software functionalities providing enhanced user convenience and functionality is also provided.

### CROSS REFERENCE TO RELATED APPLICATION

This application claims benefit to U.S. Provisional Application No. 61/063,173, filed Feb. 1, 2008 and U.S. Provisional Application No. 61/038,975, filed Mar. 24, 2008. The entire teachings of the above applications are incorporated herein by reference.

### FIELD

The present teachings relate generally to the analysis of nucleic acid samples, and in particular, but not exclusively, to a system and methods for resolving and distinguishing genetic material arising from different sources contained in a sample.

### INTRODUCTION

The need to develop increasingly automated analytical tools to perform nucleic acid sample analysis is well recognized. For example, in the forensic science community, scientists routinely process biological samples for the purposes of DNA analysis to identify composition, origin, and/or quality. Manual practices are often employed to conduct these analyses and can be time-consuming and prone to both experimental and interpretive error. Instruments capable of conducting high quality nucleic acid analysis, such as the Applied Biosystems Genetic Analyzer capillary electrophoresis systems, are increasingly relied upon to generate data for purposes of sample identification. However, there is an increasing need to extend the functionality of the data analysis component of these systems to include more sophisticated automated analysis routines to process sample data and generate highly reproducible results with minimal intervention on the part of the user.

In the context of forensic analysis, there is a need to integrate, automate, and improve the accuracy and performance of nucleic acid analysis especially where large numbers of samples must be analyzed and reported upon within a relatively short timeframe. A particular concern in forensic casework relates to resolving samples which contain mixed-populations of DNA that may arise from multiple contributors. Such samples are often encountered in criminal investigations and present significant challenges in accurately determining each of the contributor's DNA that is present within the sample. Publications describing the problems and issues associated with methods for mixed nucleic-acid sample analysis include: (1) Analysis and interpretation of mixed forensic stains using DNA STR profiling, Clayton, Whitaker, Sparkes, Gill, 1997 (2) Interpreting simple STR mixtures using allele peak areas, Gill, Sparkes, Pinchin, Clayton, Whiaker, Buckelton, 1997 (3) DNA analysis from mixed biological materials, Barbaro, Cormaci, Barbaro, 2004 (4) DNA mixtures in forensic casework: a 4-year retrospective study, Torres, Flores, Prieto, Lopez-Soto, Farfan, Carraceo, Sanz, 2003 (5) Is the 2p rule always conservative, Buckelton, Triggs, 2005 (6) LoComatioN: A software tool for the analysis of low copy number DNA profiles, Gill, Kirkham, Curran, 2006. (7) Interpreting simple STR mixtures using allele peak areas, Gill, P. et al., 1998.

### SUMMARY

In various embodiments the present teachings describe a method for DNA sample analysis comprising the steps of: (1) receiving DNA sample information comprising allelic data for a plurality of markers, each marker comprising data associated with one or more genotypes at each selected marker; (2) evaluating the allelic data for each marker and associated genotypes to classify the DNA sample information as arising from a single contributor, two contributors, or more than two contributors; (3) for DNA sample information arising from two contributors, performing an extraction routine to determine a major and minor contributor to the DNA sample information; (4) calculating statistical information for the DNA sample information used to identify the sample on the basis of the genotypes associated with each marker and provide an expected degree of confidence in the identification; and (5) outputting the statistical information used to identify the sample and the expected degree of confidence in the identification to an analyst.

In other embodiments, the present teachings describe a system DNA sample analysis comprising a data input module configured to receive DNA sample information comprising allelic data for a plurality of markers, each marker comprising data associated with one or more genotypes at each selected marker; a data processing module configured to evaluate the allelic data for each marker and associated genotypes classifying the DNA sample information as arising from a single contributor, two contributors, or more than two contributors wherein for DNA sample information arising from two contributors the data processing module performs an extraction routine to determine a major and minor contributor to the DNA sample information; and further calculates statistical information for the DNA sample information used to identify the sample on the basis of the genotypes associated with each marker and provide an expected degree of confidence in the identification; and a data output module configured to output the statistical information used to identify the sample and the expected degree of confidence in the identification to an analyst.

In still other embodiments, the present teachings describe a computer-usable medium having computer readable instructions stored thereon for execution by a processor to perform a method comprising the steps of: (1) receiving DNA sample information comprising allelic data for a plurality of markers, each marker comprising data associated with one or more genotypes at each selected marker; (2) evaluating the allelic data for each marker and associated genotypes to classify the DNA sample information as arising from a single contributor, two contributors, or more than two contributors; (3) for DNA sample information arising from two contributors, performing an extraction routine to determine a major and minor contributor to the DNA sample information; (4) calculating statistical information for the DNA sample information used to identify the sample on the basis of the genotypes associated with each marker and provide an expected degree of confidence in the identification; and (5) outputting the statistical information used to identify the sample and the expected degree of confidence in the identification to an analyst.

### BRIEF DESCRIPTION OF THE DRAWINGS

FIG. 1 shows exemplary workflow for sample analysis in accordance with the present teachings.

FIG. 2A illustrates an exemplary detailed analytical workflow for automating mixed sample analysis.

FIG. 2B illustrates an exemplary setup associated with the runtime applications and informational flow for mixture analysis.

FIG. 2C illustrates an exemplary mixture analysis pipeline in accordance with the present teachings.

FIG. 3 depicts an exemplary method for determining an expected number of contributors for a selected sample.

FIG. 4A illustrates a method for two contributor data extraction according to the present teachings.

FIG. 4B illustrates an exemplary analyst presentation of mixture analysis data in accordance with the present teachings.

FIG. 4C illustrates exemplary screenshots from a mixture analysis application in accordance with the present teachings.

FIG. 5A illustrates exemplary data associated with determination of a minor contribution at a selected locus in accordance with the present teachings.

FIG. 5B illustrates an exemplary allele dropout case at a selected locus in accordance with the present teachings.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including patents, patent applications, articles, books, treatises, and internet web pages are expressly incorporated by reference in their entirety for any purpose. In the event that one or more of the incorporated literature and similar defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art. The practice of the present teachings may employ, unless otherwise indicated, conventional techniques and descriptions of organic chemistry, polymer technology, molecular biology (including recombinant techniques), cell biology, biochemistry, and immunology, which are within the skill of the art. Such conventional techniques include oligonucleotide synthesis, hybridization, extension reaction, and detection of hybridization using a label. Specific illustrations of suitable techniques can be had by reference to the example herein below. However, other equivalent conventional procedures can, of course, also be used.

Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Gait, Oligonucleotide Synthesis: A Practical Approach 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes, Forensic DNA Typing, Second Edition: Biology, Technology, and Genetics of STR Markers, 2^{nd} Edition, John M. Butler (2005), Forensic DNA Evidence Interpretation, John S. Buckleton, Christopher M. Triggs, and Simon J. Walsh (2004) the contents of which are hereby incorporated by reference in their entirety.

The present teachings address the need to provide a reliable method of automated nucleic acid analysis including mixed-sample analysis capable of programmatic coding and software integration. The system and methods of the present teachings further provide mechanisms by which to deconvolute mixed DNA samples undergoing analysis, for example resolving two or more person mixtures into easy to interpret contributor profiles and to perform automated statistical calculations, for example CPI, CPE and/or LR. The automated analysis approach for mixed samples described herein may be part of an integrated hardware and software solution providing enhanced user convenience and functionality.

In various embodiments, the present teachings also help to reduce errors related to analysing data using multiple software and/or manual processes by integrating the analysis into a singular solution. Providing an end to end solution for automation of the analysis method in software helps to generate deterministic and reproducible results and avoids relying on subjective and error prone manual-based calculations and interpretations. The methods of the present teachings are also capable of being configured to provide more exhaustive search and identification capablilities which are highly reproducible and help alleviate time-consuming manual casework processing and labor.

As one example of the applicability of the present teachings, recent trends and requests in the forensic field have demonstrated a need for an integrated and automated method of mixed-sample deconvolution based on genotype identification and association. Mixed samples may comprise multiple different sources of contributing DNA (for example mixed perpetrator and victim DNA within a biological sample collected from a crime scene) and may be subject to various degrees of degradation. In one aspect, the methodologies of the present teachings address the fundamental challenges of analyzing these types of samples providing a user with an automated workflow which is capable of analyzing samples and presenting information regarding possible genotype combinations and probabilities of accuracy in the determination of the contributing sources to the mixed sample.

In various embodiments, the methods provided are capable of being used to automatically categorize the analyzed data and improve the efficiency of downstream analysis. In one aspect, categorization in this manner identifies a set of one or more genotypes associated with DNA recovered from a sample that may have sufficiently high probability in accuracy for inclusion in a data set used in subsequent analysis. At the same time these methods are capable of eliminating or reducing alternate/low-quality genotype calls which may adversely affect the accuracy of the analysis. As will be described in greater detail herein below, the system and methods of the present teachings may be readily integrated into existing processes/workflows and provide an analyst with the ability to dramatically improve the efficiency of identifying likely contributors to a sample mixture. For example, in forensic analysis the methods described herein may be used to define a casework workflow that is substantially more automated than existing analysis routines to provide rapid contributor identification with little or no manual data evaluation. Additionally, these methods may also provide functionality to access and evaluate multiple contributor genotype profiles allowing a reproducible and reliable mechanism by which to assess possible constituents of a given sample and their likely contributors.

Aspects of the present teachings provide software applications or modules capable of assisting a user (for example a forensic casework analyst) in the interpretation of samples which may contain mixed DNA populations. As will be described in greater detail herein below, this functionality may be configured to operate with input data obtained from another software application such as GeneMapper ID software available from Life Technologies Inc. or may be part of an embedded functionality present in the software and configured to receive and process data associated with the software.

Functionalities provided by the present teachings include, but are not limited to, performing functions such as:

Analysis of sample data and categorization as originating from a single source or contributor as well as from multiple sources or contributors (for example two sources or contributors or three or more sources).

Extraction/identification of individual or discrete sources from samples having mixed DNA populations including: separation of alleles in a mixed sample into distinct contributors, access to possible genotype combinations with functionality for automatically narrowing a given set of genotype selections to one or more likely sets to be included in a subsequent analytical workflow, and providing functionality for managing instances where at least one source/contributor to the mixed sample may be known.

Performing statistical calculations, analysis, and reporting results based on possible contributors including automated routines for identifying metrics associated with: user defined population databases, random match probabilities (RMP), combined probability of inclusion (CPI), combined probability of exclusion (CPE), and likelihood ratios (LR).

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein. Additionally, it will be appreciated that while the present teachings may refer to samples as originating from a particular source such as human DNA, the system and methods described herein are not limited to the analysis of a particular type or species of DNA. Moreover, the present teachings may be adapted for use with a variety of nucleic acid sample types and not necessarily DNA exclusively or a particular type or population of DNA.

According to the present teachings the following terms may be interpreted as follows:

Allele Frequency—The relative occurrence of a particular allele in a given population. During Mixture Analysis, the allele frequencies associated with an individual population may be used to calculate the genotype frequencies for a particular DNA profile.

C1 (Major/Major Contributor)—The DNA profile within a 2-contributor mixture sample representing the greater proportion of DNA corresponding to greater peak heights at each marker within the sample mixture. In general, for mixtures of 1:3 or higher ratios, the allele peak heights from the major contributor may be higher than the allele peak heights from the minor contributor. In situations where mixtures approaching 1:1 are analyzed, the major and minor contributors may become indistinguishable.

C2 (Minor/Minor Contributor)—In a 2-contributor mixture sample, the DNA profile representing the minority proportion of DNA corresponding to lower peak heights at each marker within the sample mixture. In general, for mixtures of 1:3 or higher ratios, the allele peak heights from the minor contributor may be lower than the allele peak heights from the major contributor and in some cases, alleles or markers may drop out. In situations where mixtures approaching 1:1 are analyzed, the major and minor contributors may become indistinguishable.

Combined Frequency—The sum of genotype frequencies at a given marker when multiple possible genotypes exist.

Contributor—An individual or originator whose DNA profile is present in a mixture sample. For example, a 2-person mixed sample may reflect contributor 1 as the major contributor or C1 (Major) and contributor 2 as the minor contributor or C2 (Minor).

CPE (Combined Probability of Exclusion)—The probability that a random person may be excluded as a possible contributor to the observed DNA mixture.

CPI (Combined Probability of Inclusion)—The probability that a random person would be included as a possible contributor to the observed DNA mixture.

Extraction—The process of separating a 2-person mixture sample into individual contributor profiles and identifying the most likely genotype combinations for each contributor profile.

F Allele—An allele designation used to indicate the potential for allelic dropout. In the Mixture Analysis application, an F allele may be included in a genotype combination if detected peaks are sufficiently low that a potential heterozygous partner to one of the detected peaks could exist below the Mixture Interpretation Threshold (MIT) within the constraints of the Peak Height Ratio (PHR) settings.

Filtering—The process of identifying eligible samples to be utilized in the Mixture Analysis routines.

Genotype Combination—A pair of genotypes that could represent the two individual contributors to a 2-person mixture sample.

Genotype Frequency—Reflects the relative occurrence of a particular genotype in a given population.

Genotype Profile—Allele designations for markers of a single-source sample or an individual contributor to a mixture sample.

Heterozygote—Individual with two different alleles at a particular marker (locus).

Homozygote—Individual with one allele at a particular marker (locus).

Inconclusive—A designation given to a marker for which the genotype has not been determined with a selected degree of certainty. In various embodiments, during Mixture Analysis, inconclusive markers may be excluded from some or all of the statistical analysis routines.

IQ (Inclusion Quality)—Reflects a quality assessment that indicates the Peak Height Ratio (PHR) Status and the Residual Status for genotype combinations.

Known Filtering—The process whereby a known genotype may be used to reduce (filter) the list of genotype combinations extracted from a 2-person mixture sample to display combinations that match the known genotype profile. During Mixture Analysis, the genotype combinations of the contributor with matches to the known contributor may be displayed in a Mixture Analysis Results Viewer.

Known Genotype Profile—Genotype of a reference sample used for comparison to a mixture sample where a known genotype is inferred (for example, an intimate body swab sample). During Mixture Analysis, the known genotype profile may be matched to one of the contributor profiles extracted from a 2-person mixture sample, and may be used to filter the genotype combinations tables to display combinations that contain the known contributor.

Known Match—A match of a known genotype to one of the contributors extracted from a 2-person mixture sample. During Mixture Analysis statistical analysis can be performed on the unknown contributor when there is a match of the known genotype to a single contributor, either C1 (Major) or C2 (Minor).

Known Matching—The process whereby a known genotype profile is compared to both of the contributor profiles extracted from a 2-person mixture sample to determine which contributor displays a match to the known.

LR (Likelihood Ratio or Hypothesis)—A ratio of the probabilities of two hypotheses that offer different explanations for the existence of the DNA profile evidence (e.g. possible contributors to the mixture sample).

Marker Inclusion Frequency—CPI/CPE Statistics that reflect the probability that a random person would be included as a possible contributor to the observed DNA mixture at a given marker.

Minimum Allele Frequency—A value that may be used in the statistical analysis of DNA profiles representing either alleles not present in the population database or alleles that have an observed allele frequency below a calculated or expected allele frequency.

Calculated using the following formula:

Minimum allele frequency=5/2n where n=number of samples for each marker in the ethnic population.

Missing Markers—Markers that are present in the mixture sample, but may not be represented in the known genotype profile.

MIT (Mixture Interpretation Threshold)—A configurable or preset setting reflected in the mixture analysis method that may be used as the minimum peak height threshold used for mixture analysis.

Mixture—A sample containing DNA from two or more contributors.

Mixture Analysis—A method or process of identifying the number of contributors to a mixture sample. In certain instances this number may reflect the minimum number of possible contributors to the mixture sample. In various embodiments, data analyzed by the mixture analysis routines is generated using one or more selected probe panels such as those provided by a AmpFISTR® kit panel (available from Life Technologies Inc.) from which is extracted potential genotypes of the contributors (e.g. 2-person mixtures) for statistical analysis. AmpFISTR® kit panels may contain components for the co-amplification of the gender markers such as Amelogenin, and fifteen short tandem repeat loci: CSF1PO, D2S1338, D3S1358, D5S818, D7S820, D8S1179, D13S317, D16S539, D18S51, D19S433, D21S11, FGA, TH01, TPOX, and vWA. Detection of these markers may be performed using Polymerase Chain Reaction (PCR) processes for DNA amplification while detection of PCR product may be accomplished on ABI PRISM® and Applied Biosystems genetic analyzer instruments following protocols established for AmpFISTR® PCR Amplification Kits. Genotypes can be assigned to samples by comparison of the sample alleles to the known alleles contained in the allelic ladder for the particular AmpFISTR® kit used. It will be appreciated that the system and methods described herein are not limited for use with any particular marker set/protocol and thus may be adapted for use with other probes and detection techniques.

Mixture Analysis Method—A collection of settings, parameters, or configurations that determine the sample segregation and extraction thresholds used by the Mixture Analysis method to analyze potential mixture samples. Data utilized by the mixture analysis methods may be provided or transferred from another software application, package, or module such as a GeneMapper® ID-X Software project.

Mixture Analysis Parameters—The heterozygote Peak Height Ratio (PHR) settings and Mixture Interpretation Threshold (MIT) as defined in the mixture analysis method, and used to perform sample segregation and extraction on selected mixture samples during Mixture Analysis.

Mixture Analysis Project—The mixture analysis results for a group of samples transferred into a Mixture Analysis tool, module, or application from another tool, module, or application such as from a GeneMapper® ID-X Software project.

Mixture Analysis Tool—In various embodiments, the Mixture Analysis Tool may be integrated into another software tool or application such as GeneMapper® ID-X Software which may also contain functionality to assist in the analysis, interpretation and statistical analysis of DNA mixtures.

Mx (Mixture Proportion)—A measure of the relative proportion of the minor contributor in a 2-person mixture sample.

PHR Status—An assessment of whether peak heights for a selected genotype combination fall above or below a Peak Height Ratio (PHR) threshold. PHR thresholds may be user-defined or predetermined in a given mixture analysis method.

Population Database—A collection of the alleles and allele frequencies obtained from a group of unrelated individuals from one or more ethnic groups. In various embodiments the Mixture Analysis methods can utilize these allele frequencies to aid in the calculation of genotype frequencies for a selected DNA profile. In one aspect, each marker within a population may be associated with a sample size (n) and may be used to determine the minimum allele frequency (calculated as 5/2n). The minimum allele frequency may be automatically assigned to any allele in each marker when an allele frequency is either not observed or below the calculated minimum allele frequency.

Profile (Sample)—The genotype (allele designations) of a sample. In various embodiments, known profiles may be imported into a mixture analysis method to compare against contributor profiles extracted from a 2-person mixture sample as part of mixture interpretation.

Profile Frequency—The estimated frequency of occurrence of a particular profile based on values from a given population database.

Reference Profile—The profile against which another profile may be compared to determine the % Match. The methods may perform pairwise comparisons to determine the direction of comparison that yields the higher % Match, then report the direction of comparison with the higher % Match. In various embodiments, one or two reference profiles (known genotypes) can be assigned to a mixture sample when calculating Likelihood Ratio (LR) statistics.

Residual—A measure of how close the observed contributor proportions for a particular genotype combination are to the expected contributor proportions for a particular 2-person mixture sample,

Residual Status—An indication of whether the calculated residual value for a genotype combination falls above or below the residual threshold (for example the residual threshold may be configured as 0.04 or another value as desired).

Residual Threshold—As defined in the Mixture Analysis method, the value above which genotype combinations are not automatically considered as possible contributors to the mixture sample.

RMP (Random Match Probability)—An expectation or probability that an individual chosen at random from the population has a DNA profile that matches the profile being compared.

Sample Segregation—The process by which samples transferred into the Mixture Analysis method from another application such as a GeneMapper® ID-X Software project are identified as containing 1, 2, or 3 or more contributors and separated into the appropriate mixture analysis workflow for each contributor category.

Sample Selection—The process by which potential mixture samples transferred into the Mixture Analysis method from another application (e.g. GeneMapper® ID-X) are selected and mixture analysis methods applied to proceed with sample segregation.

Selected Genotype Combinations Table—A table or informational set that may contain genotype combinations that are included in statistical analysis. Genotype combinations may be assigned to this table automatically or as defined within the Mixture Analysis method.

Single-Source Sample—In the Mixture Analysis method, samples originating from a single contributor. Such samples may be further defined by parameters which include: No markers that fail the peak height ratio (PHR) thresholds specified in the mixture analysis method and one marker with three called alleles. Random Match Probability and Likelihood Ratio calculations can be performed on single-source samples following sample segregation.

Statistical Analysis—The process of calculating statistics for example: Random Match Probability, Combined Probability of Inclusion, Combined Probability of Exclusion, Likelihood Ratio for a DNA profile. The Mixture Analysis method may be configured to exclude selected markers from statistical calculations. For example, an excluded marker may be Amelogenin (AMEL) marker.

Statistical Analysis Options (1 Contributor)—Displays selected genotype frequency calculation options available for use in Random Match Probability (RMP) statistical analysis of 1-contributor samples. These options may also reflect excluded markers such as the Amelogenin (AMEL) marker which are not used in statistical analyses (RMP, CPI/CPE, LR). Certain marker-specific genotype frequency calculation options may also be made available, based on allele number, for example: One allele: May use Alleles (Default), Use 2p, Inconclusive Two alleles: May use Alleles (Default), Inconclusive Three alleles: May use Min Genotype Freq (Default), Inconclusive Where: Use Alleles=Calculate the genotype frequency from the allele frequencies (use heterozygous equation [2pq] or homozygous equation [p2+p(1−p) Θ]) Use 2p=Calculate the genotype frequency from the allele frequency assuming possible allelic drop-out (use conservative frequency equation [2p]) Inconclusive=Does not calculate a genotype frequency for the marker (may consider marker as uninformative) Min Genotype Freq=Calculate the genotype frequency from the minimum genotype frequency for a tri-allelic marker (use 3/n, where n=number of samples for each marker in the ethnic population as specified in the selected population database)

Theta—A correction factor applied to the homozygous genotype frequency calculation that compensates for possible population substructure that may lead to an underestimate of the genotype frequency for the marker.

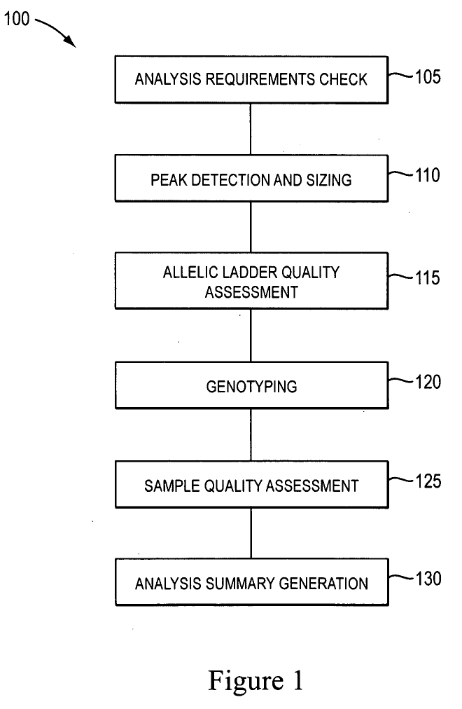

FIG. 1 shows exemplary workflow 100 for sample analysis in accordance with the present teachings. Such functionality may be integrated into a software application or package such as the GeneMapper® ID-X software application available from Life Technologies Inc. As shown in FIG. 1, the software may be configured to conduct various steps associated with a typical data analysis workflow 100 for analyzing samples and interpreting results. As will be described in greater detail herein below, these steps include determining the suitability of the data for analysis 105, performing peak data analysis and sizing 110, conducting allelic ladder or control quality assessments 115, generating genotyping calls based on allele information 120, performing sample quality assessments 125, and outputting or summarizing results to for a user 130. One beneficial aspect of this workflow is that the software may be configured to conduct these operations substantially automatically and provide an output result to the user which has been pre-evaluated/pre-screened for quality and accuracy. Such an approach reduces or eliminates user interpretation of raw data and/or avoids a user having to make detailed and time consuming analytical calculations.

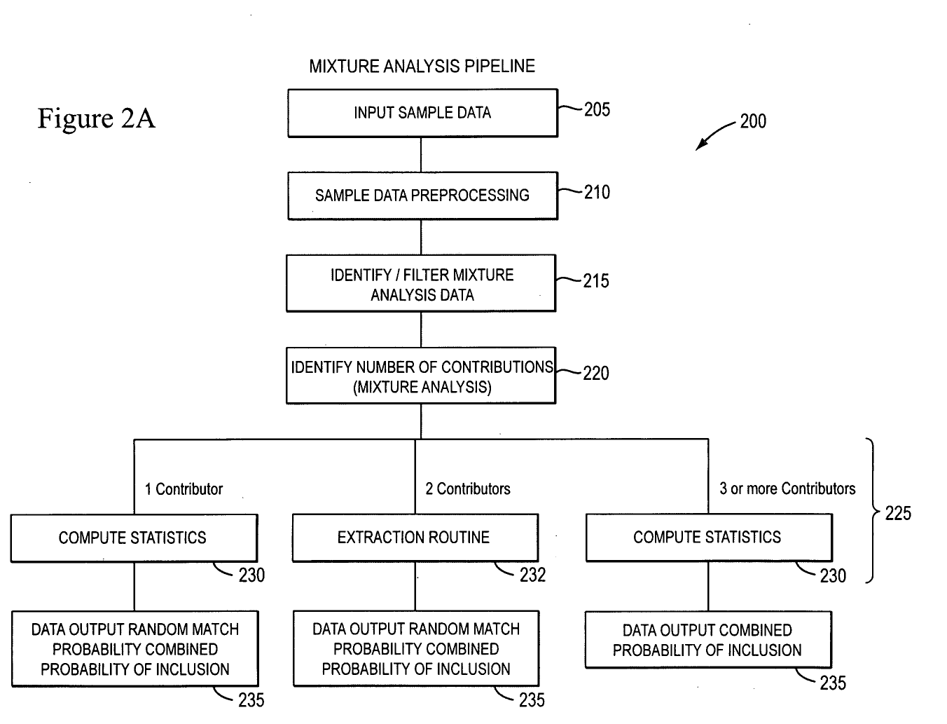

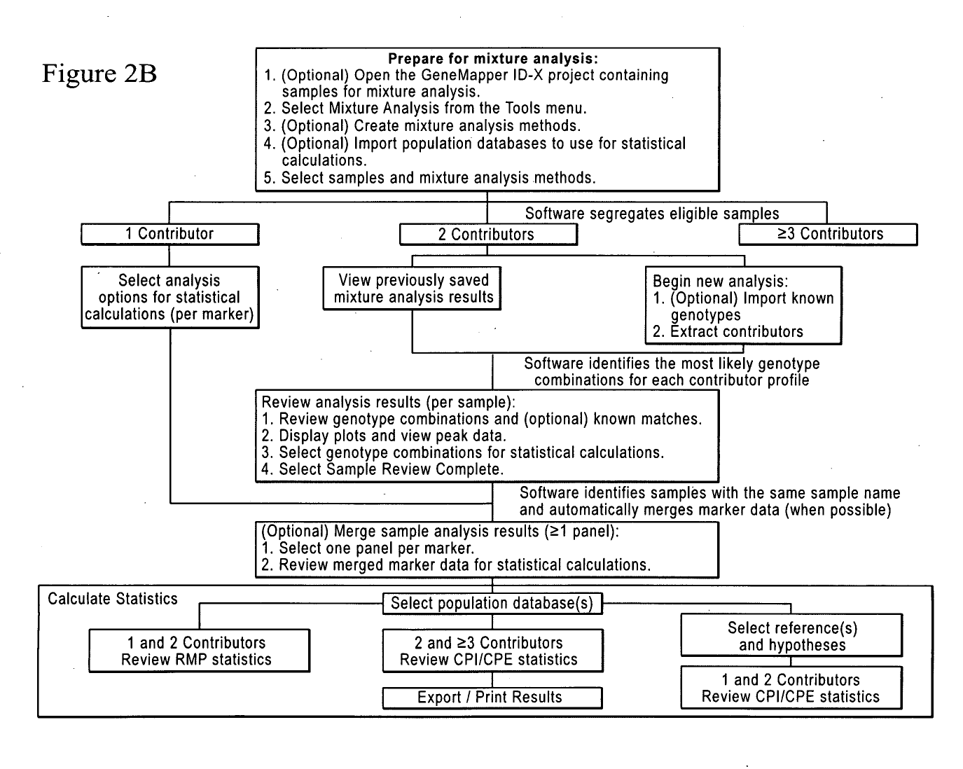

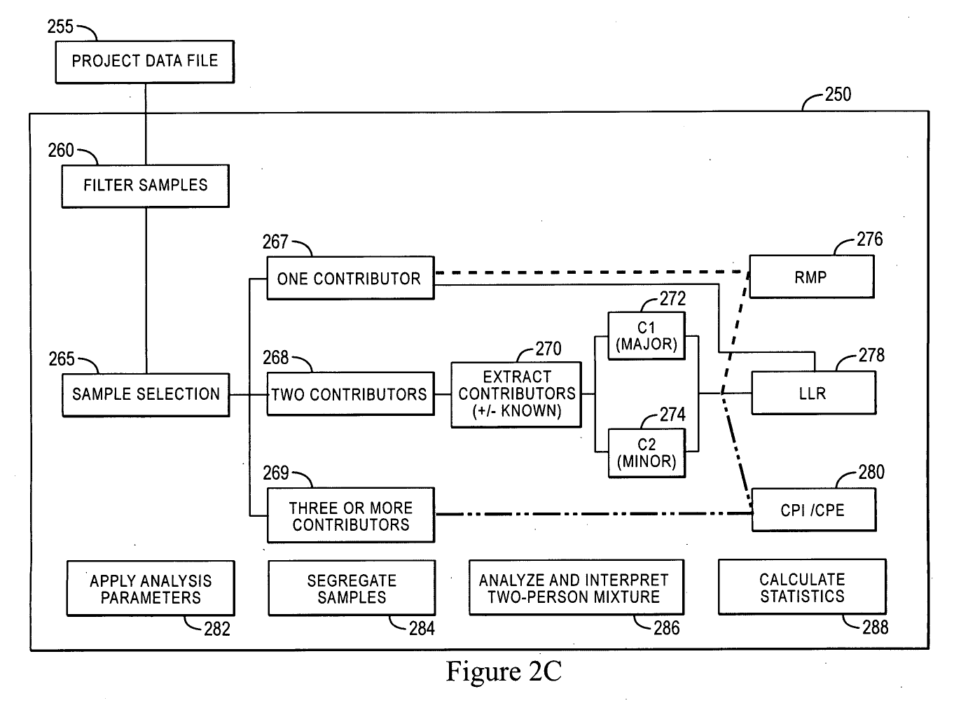

FIG. 2A illustrates a more detailed analytical workflow 200 that may be implemented by the present teachings for automating mixed sample analysis and includes the determination of the expected number of contributors to a sample. Such functionality may be invoked within another software application as a module where desired samples to be analyzed are selected by the user in step 205. In step 210 the software performs various sample data preprocessing routines which may include formatting the data, combining data, importing known, reference, or control data, and setting parameters associated with the analysis.

Input data utilized during mixture analysis may comprise project data obtained from another software module or application with the data input comprising partially analyzed, annotated, and/or edited genotype sample data, where multiple samples may be flagged for analysis. In various embodiments, the data flow takes into account both workflow and algorithmic needs. Data may be derived from an initial data input phase (for example retrieved from another module of the GeneMapper® ID-X software application) and passed through a set of processes to finally arrive at one or more statistical representations of the genotype profile extracted from the mixture.

In step 215, sample data which will be used in the mixture analysis is identified. In certain aspects, during this step 215 non-mixture data is identified. Such data may be segregated, removed, and/or flagged such that the software recognizes this data as not being part of the data set for which mixture analysis and contributor determination will be made. This non-mixture data may however be used later for purposes of quality assessment and other analyses. According to Step 215 pre-processing or conditioning operations related to the data filtered may include allelic ladder data or off ladder data. Off ladder data or peaks may comprise raw electropherogram data that does not map into specific allelic size positions from the electropherogram data using an allelic ladder and in various embodiments such data may be used to calibrate the instrument.

According to various embodiments of the present teachings, those off ladder peaks that do not fit a specific allele size may be flagged and not utilized in the mixture analysis. Samples containing such data may also be rejected due to complexities generally accepted as problematic for such an automated analysis. After samples with off ladder data are removed (if desired); a definition of the input data may be made. Such input data may comprise; a set of data collections or electropherogram results, one per marker (e.g. loci) from the DNA analysis, where each data collection may further comprise identifiers for allele positions and peak values derived from the electropherograms. In various embodiments, peak values may be obtained by measuring or calculating the maximum signal at the peak center (e.g. peak height) or measuring or calculating the peak intensity by way of computing the area under the peaks' electropherographic curve data. For additional details regarding data analysis relating to capillary electrophoresis and electropherogram peak information the reader is referred to the various references cited herein.

In various embodiments, a sample may comprise data and information relating to a selected set of markers. Typically these markers are defined by the reagent kit being used to perform the analysis. As one example, during capillary electrophoresis and analysis a set of standardized markers such as the Combined DNA Index System (CODIS) markers may be used. These markers are generally standardized for states participating in the FBI's crime-solving database. These or other markers may also be used in paternity tests and DNA fingerprint tests. Additional details and descriptions for CODIS marker information may be obtained from the following site at: http://www.fbi.gov/hq/lab/html/codisbrochure_text.htm and related pages from the FBI homepage. While there are 13 standard or core CODIS markers (14, in addition to AMEL, which indicates gender) the type and number of markers present is determined by the kit used or by analyst discretion. For example, the following markers may be used to discriminate between contributors within a sample: D3S1358, vWA, FGA, D8S1179, D21S11, D18S51, D5S818, D13S317, D7S820, D16S539, THO1, TPOX, and CSF1PO.

While it is typically important that the set of markers are selected to give both a selective measure of unique and comprehensive genes for statistical identification, the nature of the present teachings does not rely on a particular set of markers. It will be appreciated that multiple possible markers may be implemented for use with the present teachings. The type and number of markers used in connection with the present teachings is contemplated to not be limiting on the invention.

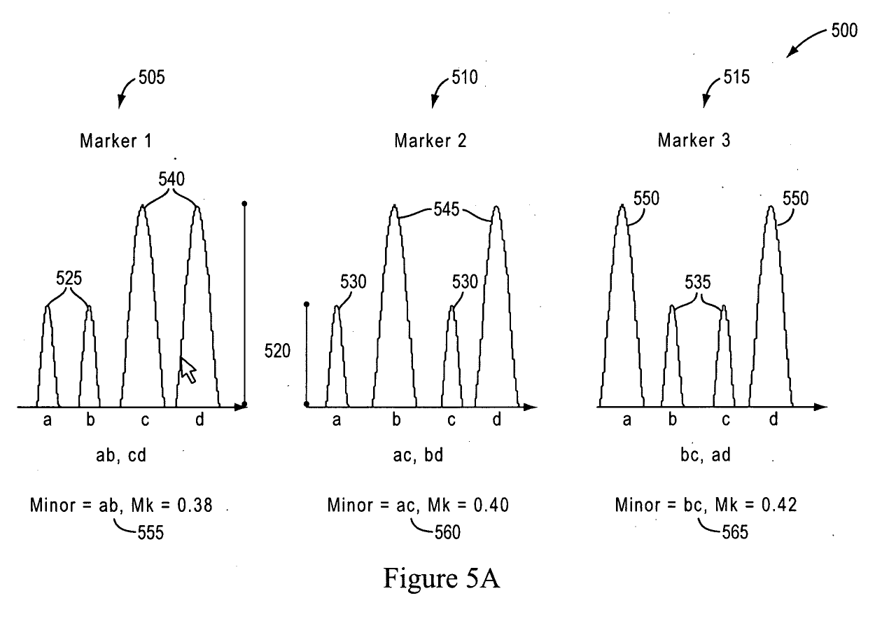

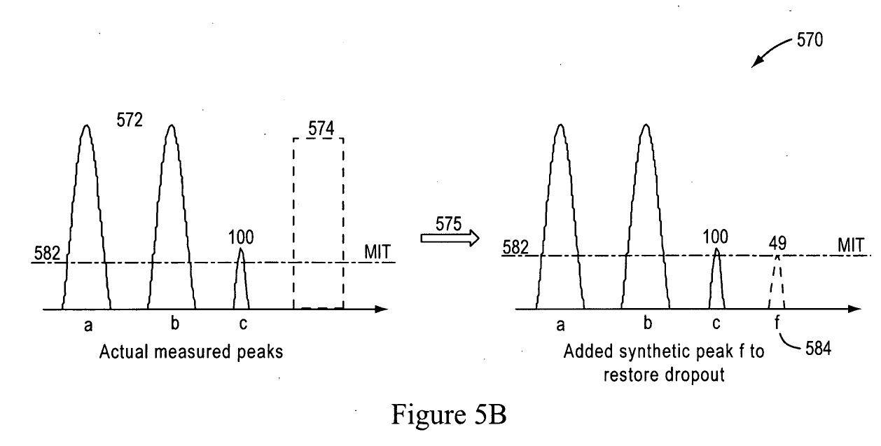

A data set for each sample may be defined as a data collection of marker information, wherein the data collection (for example one per marker) may reflect an accurate measure of the allelic data at the gene being reported. According to various embodiments of the present teachings, each sample may have some number of markers, typically in the range of approximately 5-25 markers, where each named marker may have one or more allelic peaks. Examples of the type of information generated in connection with the allelic peaks is shown in FIGS. 5A and 5B as well as other publications and references cited herein.

Exemplary filter mechanisms including peak height threshold (PHT) and peak amplitude threshold (PAT) determination may be used to reduce or eliminate electropherogram data or peaks considered below a signal-noise or detection limit. Another analysis specific threshold, is the Mixture Interpretation Threshold or Match Interpretation Threshold (MIT) which provides a measure of reliability for electropherogram peaks present in the input data collections.

In various embodiments, the peak height threshold flags or removes data upon input into the mixture analysis extraction step 220, where the individual allele data has been pre-filtered and may be considered in subsequent allele dropout scenarios. This system may be implemented with a detection step using the MIT to compare peaks against the MIT. An allele peak below the MIT may be flagged inconclusive and removed or excluded from further extraction and/or analysis processes.

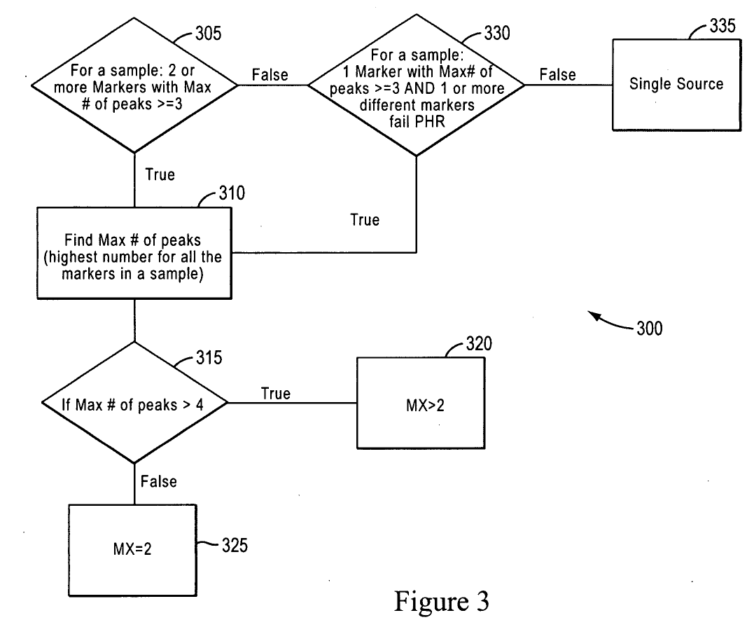

In step 220, sample data is ready for mixture analysis and evaluated to determine an expected number of contributors to the sample. A detailed explanation of the mechanisms by which a contributor number determination may be performed is provided in FIG. 3. The identified/expected number of contributors represented within the sample (for example 1, 2, or 3 as shown in FIG. 2A) may determine the subsequent actions and analysis the software performs. It will be appreciated that mixed samples may be segregated into discrete workflows for one, two, and three or more contributor mixed samples as illustrated, however, additional refinements in contributor number determination may also be made without departing from the scope of the present teachings.

In various embodiments, the mixture analysis methods of the present teachings utilize information relating to Peak Height Ratios and Mixture Interpretation Thresholds to segregate samples according to their contributor categories (e.g. 1, 2, or 3 or more contributors) and determine likely genotypes of the individual contributors to a 2-person mixture during the extraction process. Sample segregation in the aforementioned manner may be based on rules or parameters with the minimum number of expected contributors identified where 1 contributor (considered as originating from a single source) reflects samples that do not contain markers that fail the peak height ratio thresholds specified in the mixture analysis method and contain no more than 1 marker with three called alleles. Samples expected to contain 2 or more contributors may be identified by 1 or more 2-peak markers failing peak height ratio thresholds or 3 or more alleles at 2 or more markers with the maximum number of alleles not exceeding 4. Samples expected to contain 3 or more contributors may be identified by 1 or more markers with more than 4 alleles.

In step 225, the contributor number determination (for example 1 contributor and 3 or more contributors) may result in the calculation of selected statistics 230 that are output in step 235.

The type of statistical output 235 may be dependent on the contributor number to provide information most appropriate for that particular piece of data. For example, for a 1 or 2 contributor sample, data output may comprise statistics including random match probability and likelihood ratio. Alternatively, for a 2 contributor or 3 or more contributor sample, data output may comprise statistics including combined probability of inclusion/exclusion.

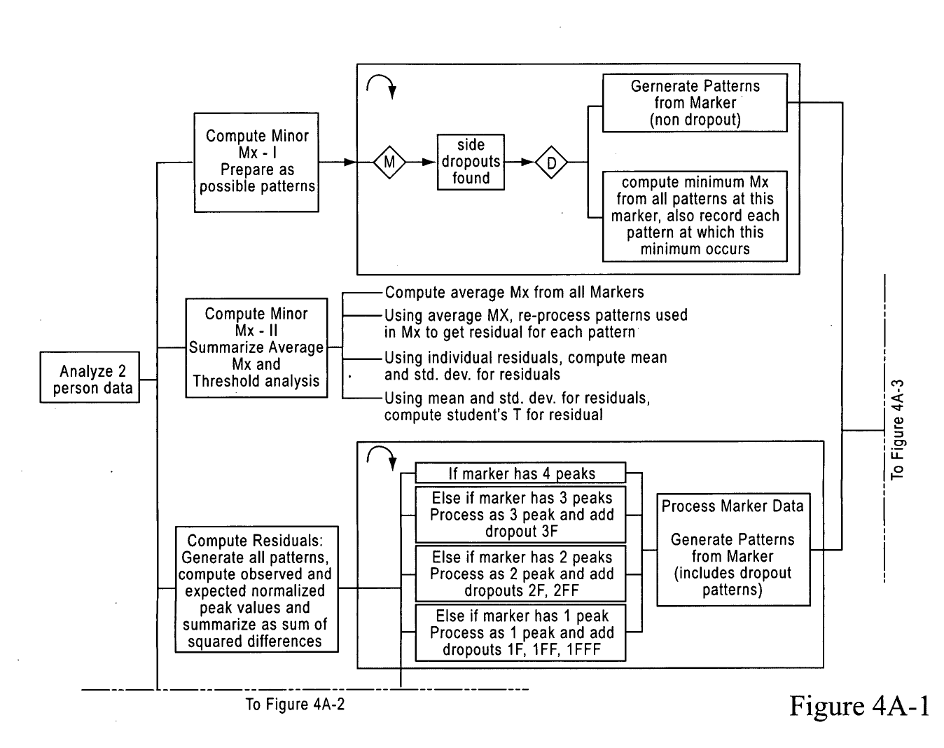

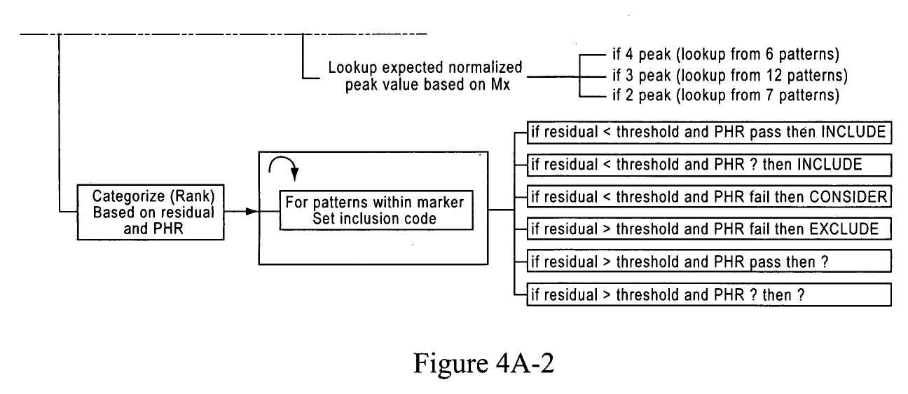

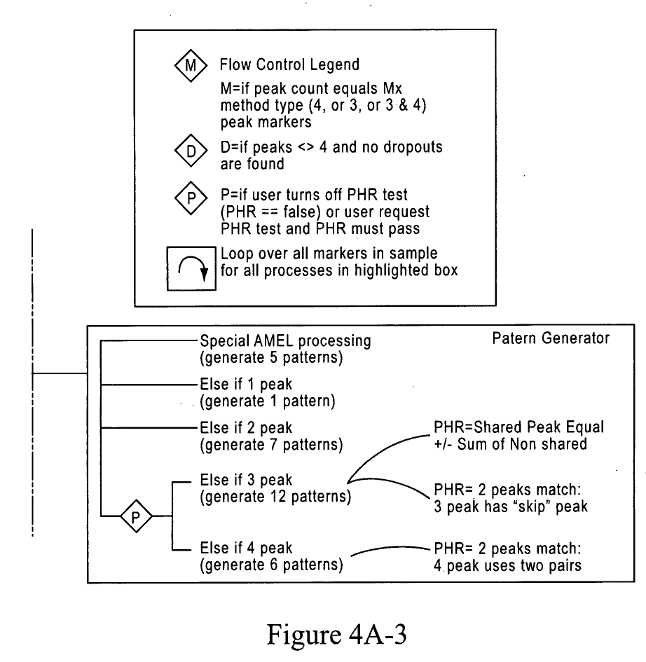

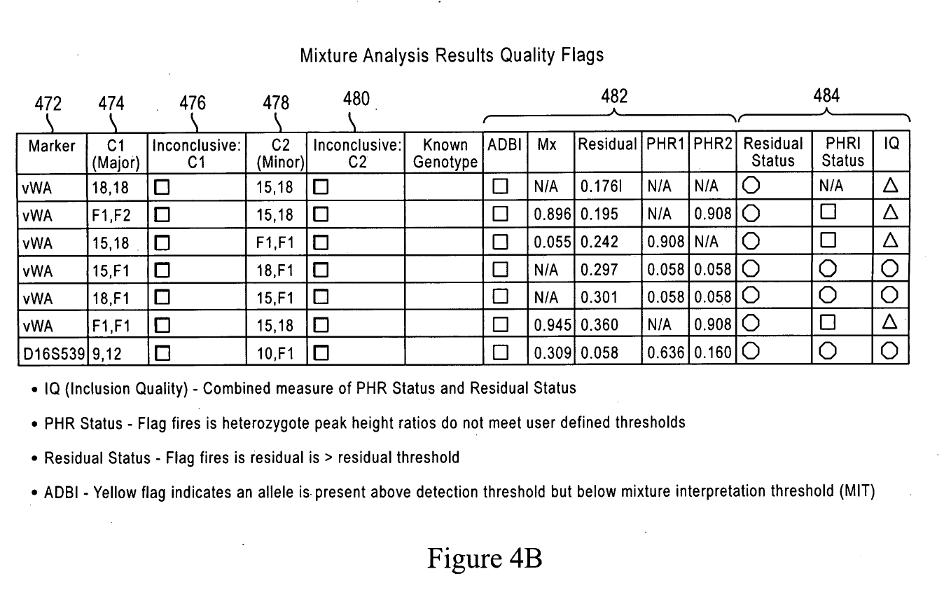

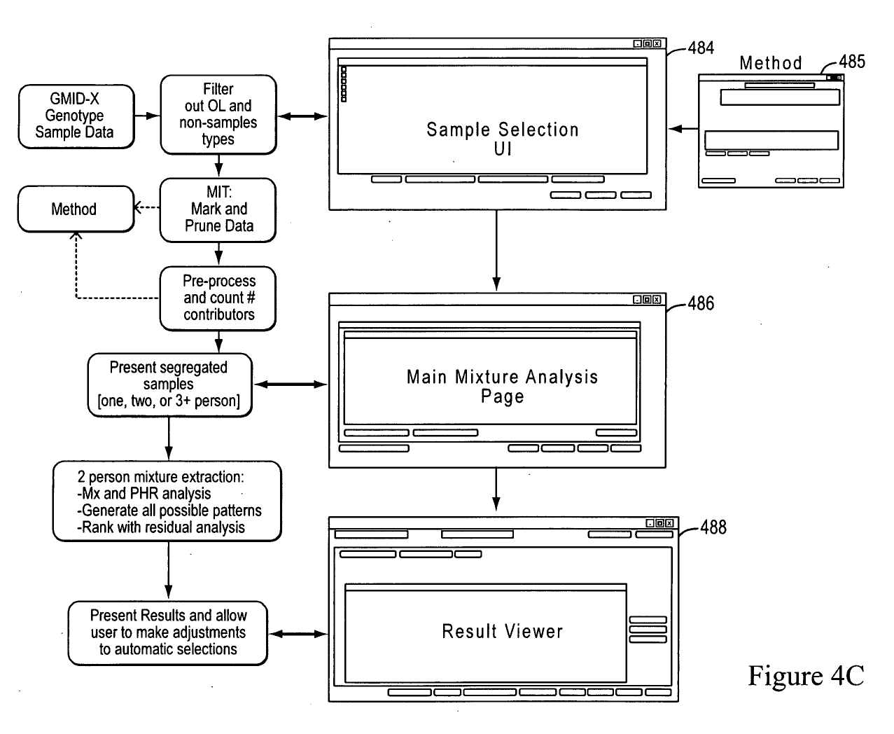

In various embodiments, where a sample is determined to comprise 2 contributors, the software may perform an additional extraction step 232 used for purposes of resolving the composition of the sample. Additional details of this extraction routine are provided with respect to FIG. 4A and its associated description. In one aspect, two contributor determinations according to the present teachings desirably identify the sources that contributed to a DNA sample of interest using known allelic/genotype information. The determination made is also capable of being associated with a score or ranking reflecting the quality and/or certainty in the identification.

Exemplary statistics calculated by the analysis methods of the present teachings include Random Match Probability, Combined Probability of Inclusion, Combined Probability of Exclusion, and Likelihood Ratio. Each of these statistical calculations may be based on allele frequency data obtained by comparison with a predefined or custom population database which has been associated with the sample data. In one aspect, an analyst can make use of an embedded or default population database such as that supplied with GeneMapper® ID-X Software or they can import their own population database information to create new selections.

It will be appreciated by one of skill in the art that these statistics desirably provide the analyst with valuable information in discriminating the sample composition as well as identifying the individual contributors to the sample. Additional details regarding these exemplary calculations as well as their use in discriminating and analyzing mixed samples will be described in greater details with reference to later figures and description.

FIGS. 2B and 2C provide more detailed views of how the method 200 illustrated in FIG. 2A may be implemented in software. FIG. 2B shows the steps associated with the runtime applications and flow of information as well as the workflow and potential points of analyst interaction. This Figure also illustrates various operations capable of being performed by the software during the analysis process. Optional aspects of the workflow are also illustrated, for example, utilizing known population databases for use in comparison against the samples of interest. It will be appreciated that these and other workflows and implementations according to the present teachings are not meant as exclusive representations of how the data may be analyzed but rather reflect various embodiments thereof.

FIG. 2C shows the mixture analysis pipeline tracking the data types and flow throughout the analysis. As previously discussed input data is processed and certain portions of this data may be excluded from the analysis improving the overall efficiency and accuracy of the system. According to this approach 250, sample data is input into the system in step 255 and subsequently filtered as previously described in step 260. In step 265, the sample to be further analyzed is determined such that after these steps have been performed, the state of the data 282 is such that it has been formatted and appropriate analysis parameters applied making the data is ready for further processing. In state 284, each sample is segregated based on the expected number of contributors to the sample. As described elsewhere, the expected number of contributors may determine the type of statistics output for analyst review. For example, in state 288 statistics may be calculated for one contributor samples which include random match probabilities and likelihood ratios. Alternatively, for three or more contributor samples, calculated statistics may include combined probability of inclusion and combined probability of exclusion.

For those samples which are expected to arise from two contributors, additional processing may take place in state 286. In step 270, the contributor profiles may be extracted and subsequently assessed to determine a major contributor 272 and minor contributor 274. Using this information, the statistical evaluation for the mixed sample may be determined as with other samples in state 288 identifying for example, random match probabilities, likelihood ratios, combined probability of inclusion, and/or combined probability of exclusion.

FIG. 3 depicts an exemplary method 300 for determining an expected number of contributors for a selected sample from a sample data collection based on electropherogram peak data as previously discussed in connection with Step 220 of FIG. 2. In various embodiments, this method 300 utilizes a decision logic configured to segregate samples into those which originate from a single source or contributor, two sources or contributors, or more than two sources or contributors. It will be appreciated that in the context of forensic analysis and casework, such a determination is of significant potential value to the analyst and may impact subsequent calculations and statistical reports generated and reviewed.

In state 305, input sample data is evaluated to determine if it conforms with two criteria including marker number and peak number. Samples that contain two or more markers with at least three peaks are further evaluated in state 310. Here a determination is made to find the relative maximum number of peaks (e.g. the highest number for all the markers in a sample). According to state 315, where the maximum number of peaks is determined to be greater than four, the sample is associated with a contributor number greater than two in state 320. For those samples having a maximum number of peaks less than or equal to four then the sample is associated with a contributor number of two in state 325.

Referring again to state 305, input sample data which does not contain at least two markers with at least three peaks each is further analyzed in state 330. In this state 330, a sample which contains a marker with a maximum number of peaks greater than two and for which at least one marker does not meet a minimum or selected peak height ratio, the value is passed to state 310 for further analysis as described previously. Those samples which do not meet the above-indicated criteria are considered as arising from a single source or contributor in state 335.

Following the exemplary method 300 for determining contributor number, once segregated, the set of samples with a minimum of two contributors may be used to perform an extraction of individual profiles. The contributors to a selected profile may be referred to as a major and minor contributor when discussed in terms of the various analysis methods used according to the present teachings. In various embodiments, for a sample which is evaluated and determined to comprise two contributing sources of DNA, there will typically be 1, 2, 3 or 4 alleles that relate to a given marker. Based on this information, the system and methods of the present teachings may leverage two significant inferences. First, is that for any locus, two alleles from the same person may be expected to have generally the same peak height/area. Heterozygous peak height ratios (PHR) may be shown to be a function of input DNA amount via validation studies. Second, established mixture proportions may generally remain consistent across loci (markers) within a sample profile.

Given the biological constraints of the input data, the present teachings provide an analysis technique for utilizing these inferences to generate pairwise profiles. These profiles may include all possible or potential genotype combinations. Using these profiles as a basis for further analysis, genotypes at each marker may be evaluated for consistency within the profile. According to the present teachings, extracting a two person mixture into a major and minor contributor is generally consistent with the typical mindset of the analyst and may be used to simplify the bookkeeping and presentation of the resulting deconvoluted results.

In various embodiments, the terms “major” and “minor” may be used as identifiers where the profile isolated as the “Major” component or contributor is unique and different from that of the “Minor” component or contributor. In one exemplary scenario when a mixture proportion is close to a 1:1 mixture of equal mass DNA materials in the sample, the system of the present teachings may be configured to produce data appropriately labeled with identified “major” and “minor” contributors. It will be appreciated that in the 1:1 case, the ordering may be somewhat arbitrary since it is expected that no individual is contributing a greater amount of genetic material or DNA. The label “major” and “minor” may still be useful in these instances however to aid in tracking marker data within the profile for subsequent statistical examination.

FIG. 4A illustrates a method 400 for two contributor data extraction according to the present teachings. This method 400 may be invoked during the operations associated with state 232 of FIG. 2 as previously discussed. The logical operations associated with data extraction are addressed in detail in FIG. 4A, where the method 400 comprises steps of which include:

Step 405 where markers to be used in the analysis are selected for the determination of the mixture proportion or Mx value.

Step 410 includes various operations where a minor mixture proportion value is determined and used to determine possible genotype allele patterns for consideration. Additionally, during this step an average Mx value is computed for the sample to be used in subsequent analysis and threshold evaluation. In various embodiments the average Mx value represents the expected mixture proportion that will be present in markers within the sample data. Another aspect to the operations performed during this step include the computation of Residuals and computation of observed and expected normalized peak values based on expected genotype allele patterns. Pattern information may also be used to categorize or rank the data based including assessments of residual values and peak height ratios.

Step 415 implements logic where peak patterns and associated markers are considered in more detail and where possible genotype combinations are computed from the input data. This may involve resolving the genotype combinations (e.g. patterns) which are represented by the mixed sample. This step may also incorporate the synthesis of peaks where an allelic dropout may occur. Additional details of pattern resolution techniques and mechanisms to address allelic dropout with synthetic peak restoration of dropout will be discussed in later sections.

Referring again to FIG. 4A, Step 420 processes markers according to the number of peaks are present. A number of approaches may be used to map major and minor contributors depending on the actual number of peaks. For example, for a four peak marker one possible mapping is provided as follows:
Minor=AB Major=CD, pattern=AB:CD
Minor=CD Major=AB, pattern=CD:AB
Minor=AC Major=BD, pattern=AC:BD
Minor=BD Major=AC, pattern=BD:AC
Minor=AD Major=BC, pattern=AD:BC
Minor=BC Major=AD, pattern=BC:AD

For a three peak marker, a number of potential ways to map the major and minor contributor exist. For example, from two types of pattern generation where there are both shared and non-shared peak patterns the following mappings may exist:

Shared Peak Patterns:
Major=AB Minor=BC, pattern=AB:BC
Major=BC Minor=AB, pattern=BC:AB
Major=AB Minor=AC, pattern=AB:AC
Major=AC Minor=AB, pattern=AC:AB
Major=AC Minor=BC, pattern=AC:BC
Major=BC Minor=AC, pattern=BC:AC

Non-Shared Peak Patterns:
Major=BC Minor=AA, pattern=BC:AA
Major=AA Minor=BC, pattern=AA:BC
Major=AC Minor=BB, pattern=AC:BB
Major=BB Minor=AC, pattern=BB:AC
Major=AB Minor=CC, pattern=AB:CC
Major=CC Minor=AB, pattern=CC:AB

For a two peak marker, a number of potential ways to map the major and minor contributor exist. For example, the following mappings may exist to map the major and minor contributors:
Major=AB Minor=AB, pattern=AB:AB
Major=AA Minor=BB, pattern=AA:BB
Major=AA Minor=AB, pattern=AA:AB
Major=BB Minor=AA, pattern=BB:AA
Major=BB Minor=AB, pattern=BB:AB
Major=AB Minor=AA, pattern=AB:AA
Major=AB Minor=BB, pattern=AB:BB

For a one peak marker, the mapping of the major and minor contributor is reflected in the following pattern:
Major=AA Minor=AA, pattern=AA:AA

For instances where an Amelogenin marker is present, the present teachings provide a number of possible ways to map the major and minor contributor reflected in the patterns should below:

When only one allele is present:
Minor=XX Major=XY, pattern=XX:XY
Minor=XY Major=XX, pattern=XY:XX
Minor=XY Major=XY, pattern=XY:XY
Minor=XX Major=XX, pattern=XX:XX

* Note * The first three patterns above result from dropout considerations

When two alleles are present:
Minor=XX Major=XY, pattern=XX:XY
Minor=XY Major=XX, pattern=XY:XX
Minor=XY Major=XY, pattern=XY:XY

Step 430 analyzes each “pattern” using the mixture proportion Mx. In various embodiments, the result is a value that measures how close the “pattern” is to the expected mixture proportion. For example, if the true mixture was AB:CD at the test marker by way of laboratory controlled mixtures, and the sample was prepared with a mixture proportion of 1 part in 4 or 1:4, then the peaks A+B/A+B+C+D would approximately be 0.25. It can be shown that a mixture proportion of AC:BD would yield a high mixture proportion and might not resemble the “pattern” since this genotype is not due to the DNA sample used in the mixture preparation. Likewise, a mixture proportion of CD:AB as simply the reverse of the AB:CD might yield a high mixture proportion and would not resemble the “pattern” known to be correct, since it may be desirable to maintain a consistent pattern relationship across markers in the sample to generate a profile for both the major and minor contributor.

Step 440 uses the expected Mx value to compute a “residual” distance from the previously determined patterns. This residual may be characterized as a numerical value that reflects how close a possible test pattern is to the expected pattern. In various embodiments, this numerical approach provides an objective, automated and reproducible method to qualify the search across possible patterns.

Step 450 analyzes each test pattern to assess whether valid Peak Height Ratios (PHR) exist. This approach provides an additional quality metric to verify the proposed pattern is valid. In various embodiments, this test automates what the laboratory looks for in peak balance.

Step 460 analyzes the residual and PHR test results used at each pattern to determine a category code that will “include” or “exclude” the pattern as likely combinations in the profile. According to the present teachings, the category code may be used to automatically segregate a selected data set into two groups including: (1) included patterns for statistical analysis and (2) excluded patterns not expected to be viable parts of either represented contributor. In various embodiments, using this approach does not necessarily suggest or conclude that a single answer or one profile for each contributor is expected, but rather a set of probable combinations as most likely genotypes in the same way a skilled human analyst might conclude as the possibilities from the input data.

Step 470 permits the system and methods of the present teachings to also be configured to allow analysts to select and deselect patterns based on exceptions and manual inspection to aid in the conclusions. Such functionality may be desirable where complexities of the input data due to sampling and instrumental artifacts might otherwise hinder a system that prevented the skilled analyst in making overrides and augmenting the automated mixture analysis.

From the aforementioned inputs and analysis the resulting profiles may be used to compute various desired statistics, including but not limited to Random Match Probability (RMP), Combined Probability of Inclusion (CPI), Combined Probability of Exclusion (CPE) and Likelihood Ratio (LR).

The following discussion provides an exemplary application of mixture analysis methods to extraction of individual contributors from 2 person mixtures. The extraction routines described herein correspond to those discussed in previous sections such as the extraction routine 232 of FIG. 2A and the pattern generation routine 415 of FIG. 4A. In various embodiments, the methods of the present teachings may be implemented in software to provide functionality for accessing possible genotype combinations and narrowing the selections by automatically categorizing the possibilities into a candidate or likely set for inclusion and subsequent analysis, while eliminating or excluding other possibilities. Evaluation of the results of contributor extraction may be simplified by the software which may be implemented using coded flags to illustrate those genotype combinations which meet the thresholds defined within the software.

FIG. 4B illustrates an exemplary output view of from data processed through the mixture analysis methods of the present teachings. In various embodiments, the data for each marker 472 under consideration is provided along with an indication of the major 474 and minor 478 alleles. Additional information may also be provided including a determination of whether the results from a particular marker are conclusive 476, 480 as well as previously described statistical results 482 and quality indicators 474 reflective of the degree of confidence in the data analysis. It will be appreciated that by presenting the data in this manner, an analyst is provided with a comprehensive and readily viewable source of information that may be used to quickly ascertain the results of the analysis without spending undo amounts of time processing and/or reviewing the details of the raw data. It will further be appreciated that the exemplary data presentation shown in FIG. 4B is but one of a variety of possible manners in which to present the mixture analysis data and that in other embodiments different types of data and/or formats may be readily implemented without departing from the scope of the present teachings.

FIG. 4C further illustrates various screenshots of the mixture analysis application. In various embodiments, screens including a sample selection interface 484, method interface 485, mixture analysis interface 486 and results viewer 488 may be implemented and which “link” into various stages of the mixture analysis methods. In various embodiments, these interfaces and screens allow the mixture analysis method to capture data and input as necessary as well as provide the analyst with the capability of viewing the progress of the analysis.

In various embodiments, separation of the alleles in a mixed sample into two distinct contributors with one or more possible genotypes at a given marker may be performed based on criteria including (a) An expected mixture proportion across a given profile and (b) expected peak height ratios for allele peaks of a given height. The expected mixture proportion across a given profile may be determined by assessing the relative contribution of the minor contributor to the mixture for 3- and 4-peak loci within a mixed profile.

As shown by the exemplary data in FIG. 5A, a determination of the minor contribution at a selected locus may be performed including calculation and averaging the minor contributor mixture proportion (Mx) across loci. An exemplary profile 500 shown in FIG. 5A comprises a profile with three 4-peak loci 505, 510, 515. For each locus, the minor contribution to the mixture is calculated based on peak height 520. As shown in this exemplary data, the peak heights 520 may vary with certain peaks being higher or of greater magnitude than other peaks. Taking the differential peak height factor into account permits for the determination of the mixture proportion resulting from the minor contributors 525, 530, 535 at each loci 505, 510, 515 relative to the major contributors 540, 545, 550.

By way of example, for the loci 505 at Marker 1, the mixture proportion of the minor contributor (Mx) 555 may be calculated as: Mx=(a+b)/(a+b+c+d) For the loci 510 at Marker 2, the mixture proportion of the minor contributor (Mx) 560 may be calculated as: Mx=(a+c)/(a+b+c+d) For the loci 515 at Marker 3, the mixture proportion of the minor contributor (Mx) 565 may be calculated as: Mx=(b+c)/(a+b+c+d)

In one aspect, to determine the minor contributor Mx 555, 560, 565 at each marker, all possible combinations may be used to find the lowest Mx value which results in the minimum or minor mixture proportion (Mx) for the locus being examined. The resulting locus-specific Mx values from all candidate loci are averaged to obtain the expected Mx (average Mx) for the mixed profile.

Upon determining the average Mx for a given profile, at each marker, all possible patterns may be generated and considered for the given set of alleles. Additionally, as previously described, allele dropout may be considered at each marker with 3 or fewer peaks. For each genotype combination, the calculated mixture proportion may be compared to the average Mx for the profile and a residual value calculated. In various embodiments, the lower the residual value, the closer the calculated mixture proportion is to the expected mixture proportion.

An exemplary allele dropout case 570 shown in FIG. 5B. As depicted in this Figure, the number of actual measured peaks (a,b,c) 572 may not necessarily correspond to an expected number of peaks. For example, for pairwise peak data representative of each allele, one expected pair may correspond to measured peaks a,b whereas peak c does not have a corresponding paired peak in an expected location 574. In one aspect, this issue may be addressed by a synthetic peak restoration process 575 to generate or “synthesize” a peak where one may be missing. Peak restoration in this manner may result in the generation of a companion peak 580 at the approximate position ‘F’ or a virtual “foreign” allele may be considered. In various embodiments, a candidate ‘F’ peak may be generated by testing various possibilities/hypotheses using Peak Height Ratio comparisons. Successful solutions to the hypothesis exist where a candidate peak qualifies as a viable match with an existing peak.

The depiction and graphical representation of the generation and inclusion of a synthetic peak f shown in FIG. 5B reflects the restoration of an exemplary dropout in accordance with the above description. One exemplary manner in which a hypothesis may be tested uses a mixture interpretation threshold (MIT) 582. For this process the MIT 582 may be set to a desired value, for example approximately 50 relative fluorescence units (rfu). Using this value as a basis for analysis in the example 3 peaks are detected above the MIT threshold. Testing each possible genotype combination which might comprise the mixture, the analysis method may take into account the possibility of the additional ‘F’ allele 584 which exists at a height of approximately 1 rfu less than the mixture interpretation threshold (MIT) to simulate a case of allele dropout. Therefore, in addition to the genotype combinations considered for the original 3 peaks (a,b,c in the examples above), a 4-peak pattern with a virtual allele ‘F’ at a peak height of MIT-1 may also be taken into consideration. A residual may then be calculated for the resulting set of combined 3 and 4 peak data and these residuals compared against a fixed threshold to divide the possible genotype combinations into two groups. In various embodiments, a “likely” and “unlikely” category may be generated for these genotype combinations. A “likely” representation may be made when the residual resides below the fixed threshold. Such a representation may be interpreted as reasonably close to the calculated mixture proportion and constitute a valid pair of genotypes to represent the individual contributors. In various embodiments, the residual threshold may be set or preconfigured in software using these methods and may be based on testing and prior experimental knowledge of mixed DNA samples.

In addition to mixture proportion, additional analysis criteria including peak height ratios (PHRs) of all possible allele combinations and displays of Pass/Fail indicators based on comparison to user-defined peak height ratio thresholds may be determined in accordance with the present teachings. These two criteria, mixture proportion and peak height ratio, may be considered together to establish an Inclusion Quality (IQ) of a given genotype combination. The resulting genotype combinations may then segregated by the IQ value, where one genotype grouping is automatically identified and included for statistical analysis and the remaining genotypes are made available for inspection but excluded from statistical calculations. Both genotype groupings may be made available for review by the analyst as well as a comparison to the underlying electropherogram data.

A further parameter for genotype combination inclusion may be employed in instances where one contributor to a mixture is known (as would be the case for a body swab sample obtained from a victim). For such instances, a known profile may be imported into the mixture analysis routine for comparison to the extracted profiles. In various embodiments, the known genotype profile may be subtracted from the data arising after the extraction of possible genotype combinations as described previously. Upon selection of a known data set, genotype combinations that have a passing IQ may be filtered such that they contain the known genotype. In instances where a known is selected, statistical calculations may be limited to only those for the unknown contributor to the mixture.

As discussed previously, various different statistical assessment approaches may be incorporated into the mixture analysis routines including but not limited to Random Match Probability (RMP), Combined Probability of Inclusion/Exclusion (CPI/E) and Likelihood Ratio (LR). These analysis approaches utilize allele frequency data obtained from predefined population databases.

The Random Match Probability assessment may be calculated for those samples categorized as arising from a single source and for selected contributors arising from a 2-person mixture extraction. In one aspect, an RMP value may be computed as previously described with a minimum allele frequency of 5/2N, where N=sample number, and for which the minimum allele frequency is utilized when the actual allele frequency does not exist in the population database or when the allele frequency is less than the minimum allele frequency.

Homozygous genotype frequencies may be calculated as (p1*p1)+p1*(1.0−p1)*θ where: p1=frequency 1 from allele 1 and θ=theta correction factor

Heterozygous genotype frequencies may be calculated as 2.0*(p1*p2) where: p1=frequency 1 from allele 1 and p2=frequency 2 from allele 2

In instances of possible allele dropout, the genotype frequency may be calculated as 2p.

In instances of locus dropout (partial profile), the locus may be rendered uninformative and a value of 1.0 is substituted for the genotype frequency.

In instances where multiple genotypes are included as possible contributors, the genotype frequencies at a given locus may be summed resulting in a combined genotype frequency for the locus. The combined genotype frequencies may be multiplied to calculate the random match probability for each contributor to the mixture.

The combined probability of inclusion/exclusion assessment may be calculated in instances involving 2 or more contributors to a mixture. For the probability of inclusion assessment the software may compute the probability of inclusion for each marker as follows:

Probability of Inclusion=Σ (Marker frequencies)²=(f₁+f₂+f₃+ . . . +f_{N})² where: Σ=sum; f₁=frequency allele 1; f₂=frequency allele 2; f₃=frequency allele 3; and N=last allele in marker data.

A combined probability of inclusion assessment may further be computed as:

Combined Probability of Inclusion=Π (Marker Probability of Inclusion₍ᵢ₎) where: Π=product and i=marker index.

For example, where the probability of inclusion for an exemplary Marker “D3”=0.01 and the probability of inclusion for and exemplary marker “D5”=0.025 the combined probability of inclusion may be determined as [(0.01)*(0.025)]=0.00025.

Therefore, if an exemplary data was associated with an ethnic group such as U.S. Hispanic, then the above example may imply that the combined probability of inclusion=0.00025 for U.S. Hispanic or stated another way 1/0.00025=4000=1 in 4 thousand U.S. Hispanics.

The combined probability of exclusion assessment may be defined as follows:

Combined probability of exclusion=1.0−Combined probability of Inclusion.

Using the above example, where combined probability of inclusion=0.00025. Combined Probability of Exclusion=1.0−0.00025=0.9997. This value may also be expressed as a percentage of the population excluded=0.99975*100=99.98%.

It will be appreciated that the illustrated implementations of the mixture analysis system and routines represent but various embodiments of how the aforementioned methods may be implemented and other programmatic schemas may be readily utilized to achieve similar results. As such, these alternative schemas are considered to be but other embodiments of the present invention. Although the above-disclosed embodiments of the present invention have shown, described, and pointed out the fundamental novel features of the invention as applied to the above-disclosed embodiments, it should be understood that various omissions, substitutions, and changes in the form of the detail of the devices, systems, and/or methods illustrated may be made by those skilled in the art without departing from the scope of the present invention. Consequently, the scope of the invention should not be limited to the foregoing description, but should be defined by the appended claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. A method for DNA sample analysis comprising:
receiving DNA sample information comprising allelic data for a plurality of markers, each marker comprising data associated with one or more genotypes at each selected marker;
evaluating the allelic data for each marker and associated genotypes to classify the DNA sample information as arising from a single contributor, two contributors, or more than two contributors;
for DNA sample information arising from two contributors, performing an extraction routine to determine a major and minor contributor to the DNA sample information;
calculating statistical information for the DNA sample information used to identify the sample on the basis of the genotypes associated with each marker and provide an expected degree of confidence in the identification; and
outputting the statistical information used to identify the sample and the expected degree of confidence in the identification to an analyst.

2. The method of claim 1 wherein the statistical information used to identify the sample is selected from the group consisting of Random Match Probability, Combined Probability of Inclusion, Combined Probability of Exclusion, and Likelihood Ratios.

3. The method of claim 1 wherein evaluating the allelic data for each marker further comprises obtaining allelic data for at least one known DNA sample for each marker and comparing the allelic data for the at least one known DNA sample to the DNA sample information.

4. The method of claim 4 wherein the DNA sample is identified on the basis of comparing the allelic data for the at least one known DNA sample to the DNA sample.

5. The method of claim 1 wherein the statistical information for the DNA sample information is further evaluated to determine if the expected degree of confidence in the identification meets at least one selected threshold wherein data which meets the at least selected threshold is reported for further analysis.

6. The method of claim 1 wherein the step of evaluating the allelic data for each marker and associated genotypes to classify the DNA sample further comprises, determining genotype patterns associated with each marker and using the genotype patterns to determine if the patterns are likely combinations for a selected DNA profile.

7. The method of claim 1 wherein the DNA sample information comprises electropherogram data and wherein the allelic data is represented by one or more peaks in the electropherogram data.

8. A system for DNA sample analysis comprising:
a data input module configured to receive DNA sample information comprising allelic data for a plurality of markers, each marker comprising data associated with one or more genotypes at each selected marker;
a data processing module configured to evaluate the allelic data for each marker and associated genotypes classifying the DNA sample information as arising from a single contributor, two contributors, or more than two contributors wherein for DNA sample information arising from two contributors the data processing module performs an extraction routine to determine a major and minor contributor to the DNA sample information; and further calculates statistical information for the DNA sample information used to identify the sample on the basis of the genotypes associated with each marker and provide an expected degree of confidence in the identification; and
a data output module configured to output the statistical information used to identify the sample and the expected degree of confidence in the identification to an analyst.

9. The system of claim 8 wherein the statistical information used to identify the sample is selected from the group consisting of Random Match Probability, Combined Probability of Inclusion, Combined Probability of Exclusion, and Likelihood Ratios.

10. The system of claim 8 wherein the data processing module further evaluates the allelic data for each marker further by obtaining allelic data for at least one known DNA sample for each marker and comparing the allelic data for the at least one known DNA sample to the DNA sample information.

11. The system of claim 10 wherein the DNA sample is identified on the basis of comparing the allelic data for the at least one known DNA sample to the DNA sample.

12. The system of claim 8 wherein the data processing module further evaluates the statistical information for the DNA sample information to determine if the expected degree of confidence in the identification meets at least one selected threshold wherein data which meets the at least selected threshold is reported for further analysis.

13. The system of claim 8 wherein the data processing module performs the evaluation of the allelic data for each marker and associated genotypes to classify the DNA sample further comprises by determining genotype patterns associated with each marker and using the genotype patterns to determine if the patterns are likely combinations for a selected DNA profile.

14. The system of claim 8 wherein the data input module is configured to receive DNA sample information comprising electropherogram data and wherein the allelic data is represented by one or more peaks in the electropherogram data.

15. A computer-usable medium having computer readable instructions stored thereon for execution by a processor to perform a method comprising:
receiving DNA sample information comprising allelic data for a plurality of markers, each marker comprising data associated with one or more genotypes at each selected marker;
evaluating the allelic data for each marker and associated genotypes to classify the DNA sample information as arising from a single contributor, two contributors, or more than two contributors;
for DNA sample information arising from two contributors, performing an extraction routine to determine a major and minor contributor to the DNA sample information;
calculating statistical information for the DNA sample information used to identify the sample on the basis of the genotypes associated with each marker and provide an expected degree of confidence in the identification; and
outputting the statistical information used to identify the sample and the expected degree of confidence in the identification to an analyst.

16. The method according to claim 15 wherein the statistical information used to identify the sample is selected from the group consisting of Random Match Probability, Combined Probability of Inclusion, Combined Probability of Exclusion, and Likelihood Ratios.

17. The method according to claim 15 wherein evaluating the allelic data for each marker further comprises obtaining allelic data for at least one known DNA sample for each marker and comparing the allelic data for the at least one known DNA sample to the DNA sample information.

18. The method according to claim 17 wherein the DNA sample is identified on the basis of comparing the allelic data for the at least one known DNA sample to the DNA sample.

19. The method according to claim 18 wherein the statistical information for the DNA sample information is further evaluated to determine if the expected degree of confidence in the identification meets at least one selected threshold wherein data which meets the at least selected threshold is reported for further analysis.

20. The method according to claim 15 further comprising the step of evaluating the allelic data for each marker and associated genotypes to classify the DNA sample further comprises, determining genotype patterns associated with each marker and using the genotype patterns to determine if the patterns are likely combinations for a selected DNA profile.

21. The method according to claim 15 wherein the DNA sample information comprises electropherogram data and wherein the allelic data is represented by one or more peaks in the electropherogram data.
